(19) United States
(12) Patent Application Publication
(10) Pub. No.: US 2024/0023802 A1
(43) Pub. Date: Jan. 25, 2024

(54) BRONCHOSCOPE AND METHOD OF USING THE SAME

(71) Applicant: KANEKA CORPORATION, Osaka-shi, Osaka (JP)

(72) Inventors: Kotaro MIYAKE, Suita-shi (JP); Shintaro OSUMI, Settsu-shi (JP); Keisuke MATSUSHITA, Tokyo (JP)

(73) Assignee: KANEKA CORPORATION, Osaka-shi, Osaka (JP)

(21) Appl. No.: 17/768,326

(22) PCT Filed: Sep. 25, 2020
(86) PCT No.: PCT/JP2020/036320

§ 371 Date: Apr. 12, 2022

(30) Foreign Application Priority Data
Oct. 16, 2019 (JP) 2019-189683

(51) Int. Cl. A61B 1/267; A61B 1/015; A61B 1/00; A61B 1/018; A61B 1/04

(52) U.S. Cl. CPC A61B 1/2676; A61B 1/015; A61B 1/00082; A61B 1/00148; A61B 1/018; A61B 1/04

(57) ABSTRACT

An object of the present invention is to provide a bronchoscope facilitating observation of a peripheral bronchus, and a method of using the same. The bronchoscope includes: a sheath having a longitudinal axis direction, the sheath having a first lumen and a second lumen each extending in the longitudinal axis direction; an endoscopic camera disposed in the first lumen; and a balloon catheter including a shaft inserted to the second lumen to be shiftable in the longitudinal axis direction and a balloon provided at a distal portion of the shaft, wherein the balloon has a pressurization lumen to be provided with fluid to receive positive pressure, and the second lumen is configured to receive negative pressure to cause a distal end of the sheath to stick to an inner wall of a bronchus.

### TECHNICAL FIELD

The present invention relates to a bronchoscope facilitating observation of a peripheral bronchus, and a method of using the same.

### BACKGROUND ART

There have been so far known various lung diseases such as chronic obstructive pulmonary disease (COPD), chronic bronchitis, and lung cancer.

Various examinations and treatments have been executed for these lung diseases, and there have been known various examination methods, treatment instruments, and the like.

For example, Patent Document 1 discloses an endoscopic catheter including a shaft, an image detector, and guide wire, as well as a balloon portion provided at a distal end of a body of the shaft. Patent Document 1 further describes that the endoscopic catheter can be moved forward to a desired position in an endolumen in a living body such as a bronchus for image diagnosis.

Patent Document 2 discloses a medical device including a long flexible shaft, an expandable portion disposed at a tip end portion of the shaft and configured to be expanded and contracted, and a removing portion configured to remove a secretion in an endolumen in a living body to outside the living body. Patent Document 3 discloses a system configured to obstruct a collateral flow channel between a target lung compartment and an adjacent lung compartment. Patent Document 4 discloses a foreign matter removal catheter including a pair of tubes configured to be inserted through a bronchoscope, the tubes each having a tip end portion provided with a balloon configured to be expanded or contracted by incoming fluid or outgoing fluid, and a nearby end portion provided with a balloon expanding syringe, the catheter configured to grip foreign matter between a tip end balloon expanded at a position beyond the foreign matter in a bronchus and a nearby balloon expanded at a position before the foreign matter. Patent Document 5 discloses a method of reducing lung capacity, the method including moving forward a bronchoscope into a reduction target lung region, and introducing a material into the target region via the bronchoscope to reduce capacity of the target region.

### RELATED ART DOCUMENTS

### Patent Documents

Patent Document 1: JP-A-2016-182302
Patent Document 2: JP-A-2017-140110
Patent Document 3: JP-A-2018-502671
Patent Document 4: JP-A-2002-000610
Patent Document 5: JP-A-2003-507130

### SUMMARY OF THE INVENTION

### Problems to be Solved by the Invention

Lung cancer sometimes occurs at a peripheral bronchus. However, conventional techniques have difficulty in inserting a bronchoscope to the peripheral bronchus that is extremely thin, for observation with use of an endoscopic camera. For example, the endoscopic catheter according to Patent Document 1 is difficult to be inserted to a peripheral bronchus due to large insertion resistance of the endoscopic catheter because a bronchoscope is inserted to a bronchus with a balloon being pressurized, and due to a large diameter of the balloon that is provided at an outer side surface of the endoscopic catheter. The conventional bronchoscopes have accordingly failed to facilitate observation of a peripheral bronchus.

The present invention has been achieved in view of the above circumstances, and it is an object of the present invention to provide a bronchoscope facilitating observation of a peripheral bronchus, and a method of using the same.

### Solutions to the Problems

The present invention provides a bronchoscope that successfully solves the above problems and is configured as follows.

[1] A bronchoscope including:
a sheath having a longitudinal axis direction, the sheath having a first lumen and a second lumen each extending in the longitudinal axis direction;
an endoscopic camera disposed in the first lumen; and
a balloon catheter including a shaft inserted to the second lumen to be shiftable in the longitudinal axis direction and a balloon provided at a distal portion of the shaft, wherein
the balloon has a pressurization lumen to be provided with fluid to receive positive pressure, and
the second lumen is configured to receive negative pressure to cause a distal end of the sheath to stick to an inner wall of a bronchus.

The distal end of the sheath can stick to the inner wall of the bronchus when the second lumen of the sheath receives negative pressure. The distal end of the sheath sticking to the inner wall of the bronchus is slid to wipe an impurity in the bronchus adhering to the endoscopic camera. Furthermore, there is provided the balloon catheter inserted to the second lumen of the sheath to be shiftable in the longitudinal axis direction, thus the user can distally extrude the balloon from the second lumen of the sheath, expand and contract the balloon, and then pull back the balloon into the second lumen. In this configuration, the peripheral bronchus can be expanded by the balloon and the bronchoscope can then be inserted to the peripheral bronchus with the balloon being contracted, thereby reducing the insertion resistance. The peripheral bronchus can thus be easily observed by wiping an impurity in the bronchus adhering to the endoscopic camera and reducing the insertion resistance of the bronchoscope.

The bronchoscope according to the present invention further has preferred aspects [2] to [10] described below.

[2] The bronchoscope according to above [1], wherein the balloon being pressurized with the pressurization lumen being provided with the fluid has an outer diameter (mm) at a center in a longitudinal axis direction of the shaft, the outer diameter being larger than an outer diameter (mm) of the sheath at the distal end of the sheath.

[3] The bronchoscope according to above [1] or [2], wherein the balloon includes a straight tube portion, and when the balloon is pressurized with the pressurization lumen being provided with the fluid, the straight tube portion has a length (mm) from a proximal end to a distal end in a longitudinal axis direction of the shaft, the length being equal to or more than the outer diameter (mm) of the balloon at the center in the longitudinal axis direction of the shaft.

[4] The bronchoscope according to above [1] or [2], wherein the balloon includes a spherical portion or a prolate spherical portion, and the spherical portion or the prolate spherical portion has a length (mm) from a proximal end to a distal end in a longitudinal axis direction of the shaft, the length being equal to or more than the outer diameter (mm) of the balloon at the center in the longitudinal axis direction of the shaft.

[5] The bronchoscope according to any one of above [1] to [4], wherein in a section perpendicular to the longitudinal axis direction of the sheath, the second lumen has an area (mm²) being 1.2 times or more an area (mm²) of a region surrounded with an outer circumferential line of the balloon being contracted.

[6] The bronchoscope according to any one of above [1] to [5], wherein the second lumen has a proximal end portion coupled with a negative pressure generator.

[7] The bronchoscope according to any one of above [1] to [6], wherein the balloon has a fixed portion fixed to the shaft and an unfixed portion not fixed thereto, and the balloon catheter includes a bar portion extending distally from a distal end of the unfixed portion.

[8] The bronchoscope according to above [7], wherein the bar portion has a length (mm) in a longitudinal axis direction, the length being 0.5 times or more the outer diameter (mm) of the balloon at the center in the longitudinal axis direction of the shaft, the balloon being pressurized with the pressurization lumen being provided with the fluid.

[9] The bronchoscope according to above [7] to [8], wherein the bar portion has a distal end portion including a curved portion having a curvature radius of 0.1 mm or more and 10 mm or less in a sectional view in a longitudinal axis direction.

[10] The bronchoscope according to any one of above [1] to [9], wherein the balloon is a noncompliant balloon or a semicompliant balloon.

The present invention further includes a method of using the bronchoscope as in [11] below, as well as preferred aspects thereof [12] to [15].

[11] A method of using the bronchoscope according to any one of above [1] to [10], the method including applying negative pressure to the second lumen to cause the distal end of the sheath to stick to the inner wall of the bronchus.

[12] The method of using the bronchoscope according to above [11], the method including sliding the distal end of the sheath sticking to the inner wall of the bronchus, to remove an impurity in the bronchus adhering to a distal end of the endoscopic camera.

[13] The method of using the bronchoscope according to above [11] or [12], wherein sticking to the inner wall of the bronchus is executed while negative pressure is applied to the pressurization lumen of the balloon.

[14] The method of using the bronchoscope according to any one of above [11] to [13], the method including:
distally extruding the balloon from a distal end of the second lumen;
applying positive pressure to the balloon to expand the balloon;
applying negative pressure to the balloon to contract the balloon;
pulling back the balloon into the second lumen; and
observing with use of the endoscopic camera.

[15] The method of using the bronchoscope according to above [14], the method including distally pushing the bronchoscope after the balloon is pulled back into the second lumen.

### Effects of the Invention

According to the present invention, the configurations described above enable provision of a bronchoscope facilitating observation of a peripheral bronchus.

### BRIEF DESCRIPTION OF THE DRAWINGS

FIG. 1 is a perspective view of a bronchoscope according to an embodiment of the present invention.

FIG. 2 is a side view (partially sectional view) depicting a method of using the bronchoscope according to the embodiment of the present invention.

FIG. 3 is a side view depicting the method of using the bronchoscope according to the embodiment of the present invention.

FIG. 4 is a side view (partially sectional view) depicting the method of using the bronchoscope according to the embodiment of the present invention.

FIG. 5 is a plan view of a distal end of a sheath included in the bronchoscope according to the embodiment of the present invention.

FIG. 6 is a side view depicting the method of using the bronchoscope according to the embodiment of the present invention.

FIG. 7 is a side view depicting the method of using the bronchoscope according to the embodiment of the present invention.

FIG. 8 is a side view depicting the method of using the bronchoscope according to the embodiment of the present invention.

FIG. 9 is a side view depicting the method of using the bronchoscope according to the embodiment of the present invention.

FIG. 10 is a sectional view in a longitudinal axis direction of a region R illustrated in FIG. 4.

FIG. 11 is a different exemplary sectional view in the longitudinal axis direction of the region R illustrated in FIG. 4.

FIG. 12 is a different exemplary sectional view in the longitudinal axis direction of the region R illustrated in FIG. 4.

FIG. 13 is a different exemplary sectional view in the longitudinal axis direction of the region R illustrated in FIG. 4.

FIG. 14 is a different exemplary sectional view in the longitudinal axis direction of the region R illustrated in FIG. 4.

FIG. 15 is a different exemplary sectional view in the longitudinal axis direction of the region R illustrated in FIG. 4.

### MODE FOR CARRYING OUT THE INVENTION

More specific description will now be made to the following embodiment of the present invention. The present invention should not be limited by the following embodiment, but can obviously be implemented with appropriate modifications within a range applicable to purport of the above and below description. Such modifications will be all included in the technical scope of the present invention. Some of the drawings may not include reference signs of members for convenience, in which case the specification or any other appropriate drawing should be referred to. Various members in the drawings may not be depicted in actual sizes, because contribution to comprehension of the features of the present invention is prioritized in the drawings.

A bronchoscope according to the present invention includes: a sheath having a longitudinal axis direction, the sheath having a first lumen and a second lumen each extending in the longitudinal axis direction; an endoscopic camera disposed in the first lumen; and a balloon catheter including a shaft inserted to the second lumen to be shiftable in the longitudinal axis direction and a balloon provided at a distal portion of the shaft, in which the balloon has a pressurization lumen to be provided with fluid to receive positive pressure, and the second lumen is configured to receive negative pressure to cause a distal end of the sheath to stick to an inner wall of a bronchus.

In the bronchoscope according to the present invention, the distal end of the sheath can stick to the inner wall of the bronchus when the second lumen of the sheath receives negative pressure. The distal end of the sheath sticking to the inner wall of the bronchus is slid to wipe an impurity in the bronchus adhering to the endoscopic camera. The bronchoscope according to the present invention further includes the balloon catheter inserted to the second lumen of the sheath to be shiftable in the longitudinal axis direction, thus the user can distally extrude the balloon from the second lumen of the sheath, expand and contract the balloon, and then pull back the balloon into the second lumen. In this configuration, the peripheral bronchus can be expanded using the balloon and the bronchoscope can then be inserted to the peripheral bronchus with the balloon being contracted, thereby reducing the insertion resistance. The peripheral bronchus can thus be easily observed by wiping an impurity in the bronchus adhering to the endoscopic camera and reducing the insertion resistance of the bronchoscope.

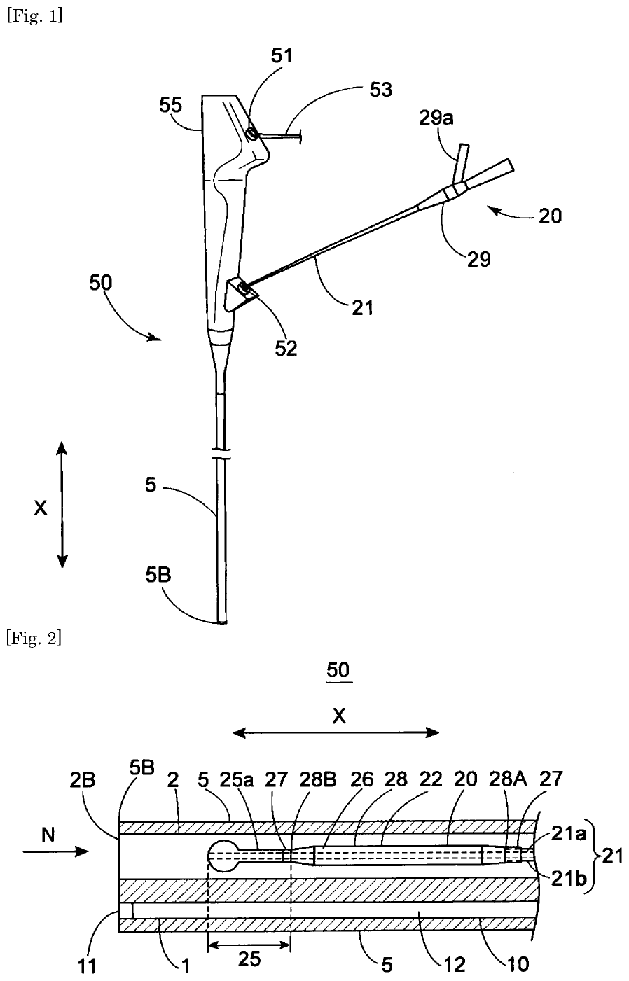

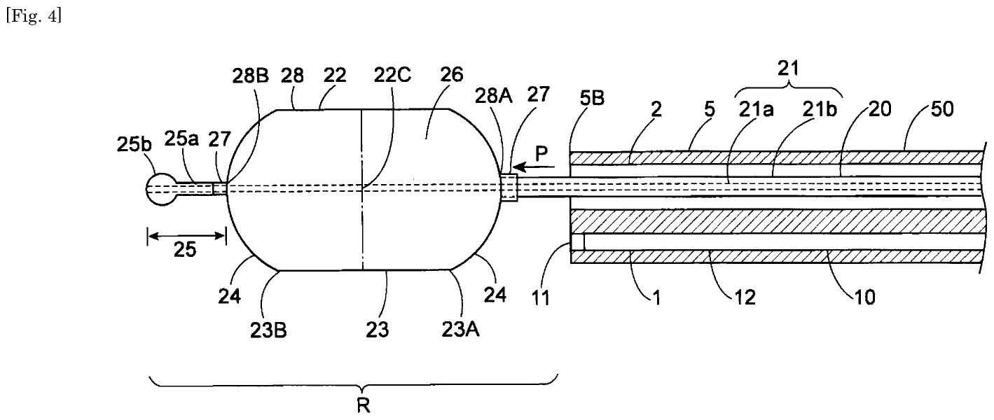

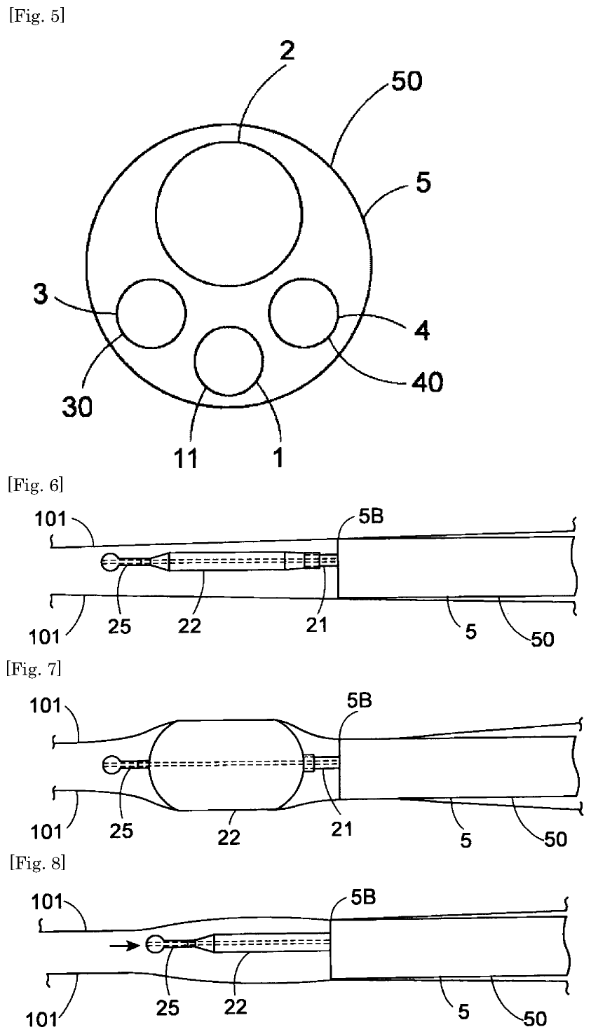

With reference to FIGS. 1 to 15, description is made hereinafter to a bronchoscope and a method of using the same according to the embodiment of the present invention. FIG. 1 is a perspective view of the bronchoscope according to the embodiment of the present invention. FIGS. 2 to 4 and 6 to 9 are side views depicting the method of using the bronchoscope according to the embodiment of the present invention. FIGS. 2 and 4 include hatched portions indicating a section of a sheath. FIG. 4 further includes dashed lines indicating an outer diameter of a balloon at a center in a longitudinal axis direction of a shaft. FIG. 5 is a plan view of a distal end of the sheath in the bronchoscope according to the embodiment of the present invention. FIG. 10 is a sectional view in a longitudinal axis direction of a region R illustrated in FIG. 4. FIGS. 11 to 15 are different exemplary sectional views in the longitudinal axis direction of the region R illustrated in FIG. 4.

FIGS. 1 and 2 depict a bronchoscope 50 including a sheath 5 having a first lumen 1 and a second lumen 2 each extending in a longitudinal axis direction X. The sheath 5 has the first lumen 1 and the second lumen 2 and is not particularly limited. The sheath 5 has an outer diameter that is preferably 6 mm or less and is more preferably 3 mm or less. The sheath 5 thus configured can be easily inserted to a peripheral bronchus having an inner diameter of 2 mm or less. Meanwhile, the outer diameter of the sheath 5 has a lower limit that is preferably 1 mm or more and is more preferably 2 mm or more in consideration of a size of an endoscopic camera 10 to be described later, and the like. The sheath 5 is made of a material, examples of which include a polyamide resin, a polyester resin, a polyurethane resin, a polyolefin resin, a vinyl chloride resin, a silicone resin, a fluororesin, an epoxy resin, and natural rubber. The sheath 5 may be made of only one of these materials, or two or more of these materials.

As depicted in FIG. 2, the first lumen 1 is provided therein with the endoscopic camera 10. The endoscopic camera 10 is preferably fixed to the first lumen 1 so as not to move in the longitudinal axis direction X. The endoscopic camera 10 is not particularly limited, and exemplarily includes an object lens 11 and an image transmitter 12. Examples of the image transmitter 12 include image guide fiber constituted by optical fiber, and a relay lens constituted by a large number of lenses. The endoscopic camera 10 acquires image information that is transmitted to a display device via a connector portion 53 of the bronchoscope 50 depicted in FIG. 1 or the like, and the display device displays the image information.

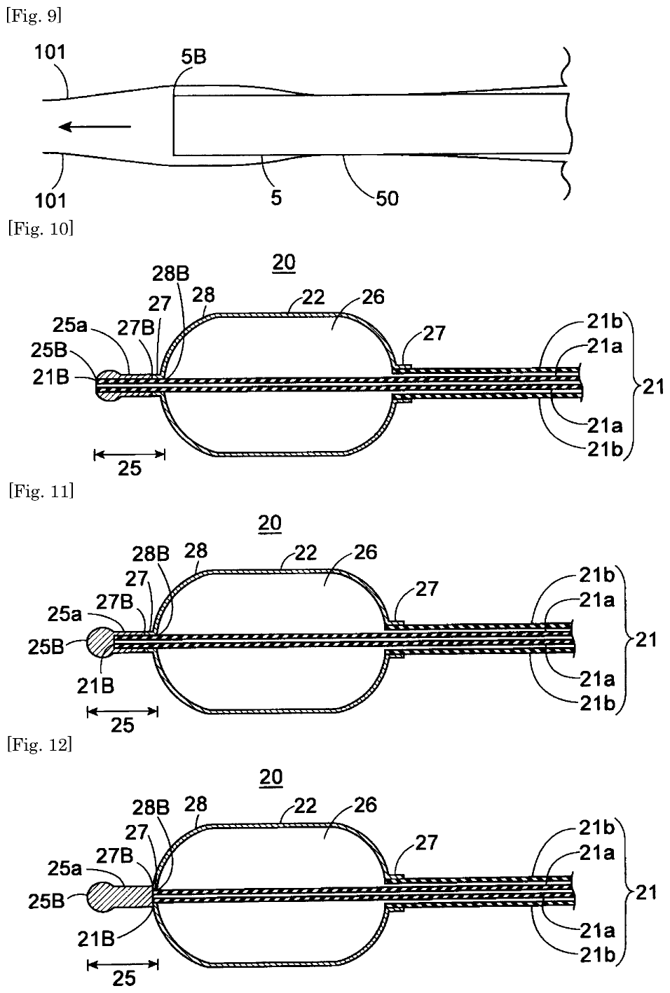

As depicted in FIG. 2, a balloon catheter 20, which includes a shaft 21 and a balloon 22 provided at a distal portion of the shaft 21, is inserted to the second lumen 2 of the sheath 5 so as to be shiftable in the longitudinal axis direction X. Specifically, the balloon catheter 20 is inserted to the second lumen 2 of the sheath 5 via an insertion hole 52 of the bronchoscope 50 depicted in FIG. 1, and the balloon catheter 20 is not fixed so as to be shiftable in the longitudinal axis direction X. Furthermore, the balloon 22 includes a pressurization lumen 26 to be provided with fluid to receive positive pressure. In the bronchoscope 50 thus configured, the balloon 22 inserted to the second lumen 2 can be distally pushed from the second lumen 2 to expand the balloon 22 as depicted in FIGS. 2, 4, 6, and 7, and the balloon 22 can be contracted and then be pulled back into the second lumen 2 as depicted in FIG. 8. Furthermore, the sheath 5 can be inserted to a peripheral bronchus 101 being expanded, in a state where the balloon 22 is accommodated in the second lumen 2 as depicted in FIG. 9.

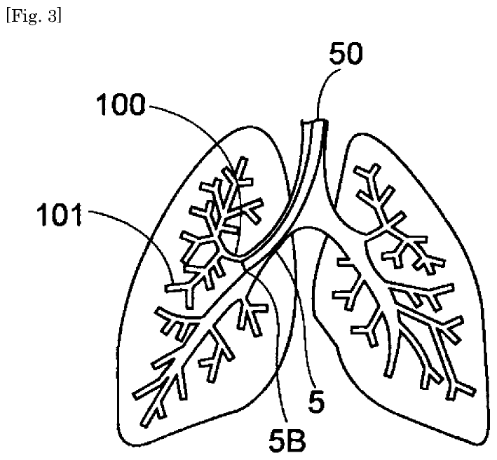

When the second lumen 2 receives negative pressure N as depicted in FIG. 2, a distal end 5B of the sheath 5 sticks to an inner wall of a bronchus 100 as depicted in FIG. 3. Furthermore, the distal end 5B of the sheath 5 sticking to the inner wall of the bronchus 100 is slid while being pushed against the inner wall of the bronchus 100, to wipe an impurity in the bronchus 100 adhering to the object lens 11 of the endoscopic camera 10. This facilitates observation of the peripheral bronchus 101.

As depicted in FIG. 4, the balloon 22 being pressurized with the pressurization lumen 26 being provided with the fluid has an outer diameter (mm) at a center 22C in a longitudinal axis direction of the shaft 21, and the outer diameter is preferably larger than the outer diameter (mm) of the sheath 5 at the distal end 5B of the sheath 5. The configuration can easily make an inner diameter of peripheral bronchus 101 larger than the outer diameter of the sheath 5, for easy insertion of the sheath 5 to the peripheral bronchus 101. The outer diameter (mm) of the balloon 22 at the center 22C in the longitudinal axis direction is more preferably 1.2 times or more the outer diameter (mm) of the sheath 5 at the distal end 5B of the sheath 5, further preferably 1.5 time or more, still further preferably 2.0 times or more, and particularly preferably 2.5 times or more. Meanwhile, the outer diameter (mm) of the balloon 22 at the center 22C in the longitudinal axis direction is preferably set to be 5.0 times or less the outer diameter (mm) of the sheath 5 at the distal end 5B of the sheath 5, in order to easily avoid damage to the peripheral bronchus 101. The outer diameter is thus preferably 5.0 times or less, more preferably 4.5 times or less, further preferably 4.0 times or less, and still further preferably 3.5 times or less.

The balloon 22 is not particularly limited in terms of its shape, and can include a straight tube portion 23 and a tapered portion 24 as depicted in FIG. 4, or can include a spherical portion or a prolate spherical portion. The balloon 22 can more easily expand the peripheral bronchus 101 with a larger contact area with an inner wall of the peripheral bronchus 101. Accordingly, the balloon 22 preferably includes the straight tube portion or the prolate spherical portion, and more preferably includes the straight tube portion. Meanwhile, in a case where the negative pressure N needs to be increased to stick the distal end 5B of the sheath 5 to the inner wall of the bronchus 100, the balloon 22 preferably includes the spherical portion because the length being shorter in the longitudinal axis direction is more likely to prevent expansion of the balloon 22 by the negative pressure N.

In the case where the balloon 22 includes the straight tube portion 23 and is pressurized with the pressurization lumen 26 being provided with the fluid, the straight tube portion 23 has a length (mm) from a proximal end 23A to a distal end 23B in the longitudinal axis direction of the shaft 21, and the length is preferably equal to or more than the outer diameter (mm) of the balloon 22 at the center 22C in the longitudinal axis direction of the shaft 21. This facilitates wide expansion of the peripheral bronchus 101, for easy insertion of the sheath 5 to the peripheral bronchus 101. Accordingly, the length (mm) from the proximal end 23A to the distal end 23B is preferably 1.1 times or more the outer diameter (mm) of the balloon 22 at the center 22C in the longitudinal axis direction, and more preferably 1.2 times or more. Meanwhile, the length (mm) from the proximal end 23A to the distal end 23B has an upper limit not particularly limited, and can be exemplarily five times or less the outer diameter (mm) of the balloon 22 at the center 22C in the longitudinal axis direction.

In the case where the balloon 22 includes the spherical portion or the prolate spherical portion, the spherical portion or the prolate spherical portion has a length (mm) from a proximal end to a distal end in the longitudinal axis direction of the shaft 21, and the length is preferably equal to or more than the outer diameter (mm) of the balloon 22 at the center 22C in the longitudinal axis direction of the shaft 21. This facilitates wide expansion of the peripheral bronchus 101, for easy insertion of the sheath 5 to the peripheral bronchus 101. Accordingly, the length (mm) from the proximal end to the distal end is more preferably 1.1 times or more the outer diameter (mm) of the balloon 22 at the center 22C in the longitudinal axis direction, and further preferably 1.2 times or more. Meanwhile, the length (mm) from the proximal end to the distal end has an upper limit not particularly limited, and can be exemplarily five times or less the outer diameter (mm) of the balloon 22 at the center 22C in the longitudinal axis direction.

The balloon 22 is preferably a noncompliant balloon or a semicompliant balloon. These balloons are each hard to be expanded when fluid injection pressure has a predetermined value or more. This can facilitate avoidance of damage to the peripheral bronchus 101 due to excessive expansion of the balloon 22. The semicompliant balloon is more preferred because the semicompliant balloon easily achieves both safety and expansion of the peripheral bronchus 101. The noncompliant balloon has 4% or less as an outer diameter change rate of the balloon 22 at the center 22C in the longitudinal axis direction upon pressurization from 50% of specified pressure (pressure having a nominal value=nominal pressure) of the balloon to the specified pressure. In contrast, the semicompliant balloon has more than 4% and 15% or less as the outer diameter change rate of the balloon 22 at the center 22C in the longitudinal axis direction upon pressurization from 50% of specified pressure (pressure having a nominal value=nominal pressure) of the balloon to the specified pressure. The specified pressure is preferably 2 atm or more and 20 atm or less, and more preferably 3 atm or more and 10 atm or less.

The balloon 22 may be made of a resin, examples of which include a polyamide resin, a polyester resin, a polyurethane resin, a polyolefin resin, a vinyl chloride resin, a silicone resin, and natural rubber. The balloon 22 may be made of only one of these resins, or two or more of these resins.

The balloon 22 may alternatively include a reinforcing material provided on an outer side surface of a resin layer made of the resin, for improvement in dimensional safety against expansion pressure. Examples of the reinforcing material include a fiber material. The reinforcing material may be provided entirely on the outer side surface of the resin layer without any gap, or may be provided only partially on the outer side surface of the resin layer. Examples of the fiber material adopted as the reinforcing material include polyarylate fiber, aramid fiber, ultrahigh molecular weight polyethylene fiber, PBO fiber, and carbon fiber. Each of these fiber materials may be a monofilament or a multifilament.

The fluid injected to the pressurization lumen 26 of the balloon 22 is not particularly limited, and can be liquid or gas. The fluid may be pressurized with use of a syringe, an indeflator, a pump, or the like before being injected to the pressurization lumen 26.

The number the balloon 22 is not limited to one, and there may be two or more balloons. The balloon 22 smaller in the number more easily enters and leaves the second lumen 2, so that there is preferably provided one balloon 22.

In a section perpendicular to the longitudinal axis direction of the sheath 5, the second lumen 2 preferably has an area (mm²) being 1.2 times or more an area (mm²) of a region surrounded with an outer circumferential line of the balloon 22 being contracted. Also in a state where the balloon 22 is disposed in the second lumen 2, the negative pressure N can thus be applied to the second lumen 2 to easily stick the distal end 5B of the sheath 5 to the inner wall of the bronchus 100. Accordingly, the area (mm²) of the second lumen 2 is more preferably 1.3 times or more the area (mm²) of the region surrounded with the outer circumferential line of the balloon 22 being contracted, and further preferably 1.4 times or more. Meanwhile, the area (mm²) of the second lumen 2 is preferably eight times or less the area (mm²) of the region surrounded with the outer circumferential line of the balloon 22 being contracted, for easy avoidance of damage due to excessive sticking of the distal end 5B of the sheath 5 to the inner wall of the bronchus 100. The area is more preferably 6.5 times or less, and further preferably 5 times or less.

As depicted in FIGS. 2, 4, and 10, the shaft 21 may include an inner tube 21a and an outer tube 21b. The inner tube 21a can be utilized as an insertion path for guide wire or the like. In FIGS. 2, 4, and 10, the balloon 22 has a distal end portion fixed to the inner tube 21a at a distal fixed portion 27, and has a proximal end portion fixed to the outer tube 21b at a proximal fixed portion 27. Such a configuration can utilize a space between the inner tube 21a and the outer tube 21b as a flow path for fluid injection, and can further utilize a space between the inner tube 21a and the balloon 22 as the pressurization lumen 26. The inner tube 21a has a distal end 21B that may be positioned distally to a distal end 27B of the distal fixed portion 27 of the balloon 22 as depicted in FIGS. 10 and 11, or may be aligned with the distal end 27B in the longitudinal axis direction as depicted in FIG. 12.

In a case where the balloon catheter 20 includes a bar body 25a to be described later, the distal end 21B of the inner tube 21a may be aligned with a distal end 25B of the bar body 25a in the longitudinal axis direction as depicted in FIG. 10, may be positioned proximally to the distal end 25B of the bar body 25a and distally to the distal end 27B of the distal fixed portion 27 of the balloon 22 as depicted in FIG. 11, or may be aligned with the distal end 27B in the longitudinal axis direction as depicted in FIG. 12. That is, the inner tube 21a has a distal end portion that may penetrate the bar body 25a in the longitudinal axis direction as depicted in FIG. 10, may be incorporated in the bar body 25a as depicted in FIG. 11, or may be joined to be adjacent to the bar body 25a as depicted in FIG. 12.

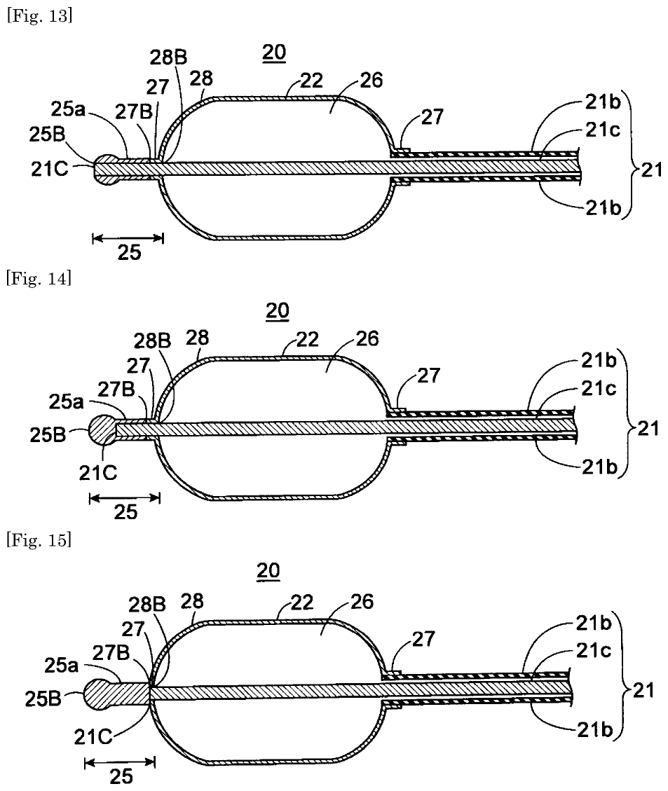

As depicted in FIG. 13, the shaft 21 may include the outer tube 21b and a linear body 21c disposed in the outer tube 21b. Accordingly, the distal end portion of the balloon 22 can be fixed to the linear body 21c at the distal fixed portion 27, and the proximal end portion of the balloon 22 can be fixed to the outer tube 21b at the proximal fixed portion 27. Such a configuration can utilize a space between the linear body 21c and the outer tube 21b as the flow path for fluid injection, and can further utilize a space between the linear body 21c and the balloon 22 as the pressurization lumen 26. The linear body 21c has a distal end 21C that may be positioned distally to the distal end 27B of the distal fixed portion 27 of the balloon 22 as depicted in FIGS. 13 and 14, or may be aligned with the distal end 27B in the longitudinal axis direction as depicted in FIG. 15.

In a case where the balloon catheter 20 includes the bar body 25a to be described later, the distal end 21C of the linear body 21c may be aligned with the distal end 25B of the bar body 25a in the longitudinal axis direction as depicted in FIG. 13, may be positioned proximally to the distal end 25B of the bar body 25a and distally to the distal end 27B of the distal fixed portion 27 of the balloon 22 as depicted in FIG. 14, or may be aligned with the distal end 27B in the longitudinal axis direction as depicted in FIG. 15. That is, the linear body 21c has a distal end portion that may penetrate the bar body 25a in the longitudinal axis direction as depicted in FIG. 13, may be incorporated in the bar body 25a as depicted in FIG. 14, or may be joined to be adjacent to the bar body 25a as depicted in FIG. 15.

Though not depicted, the shaft 21 may alternatively be constituted by the linear body 21c without including the inner tube 21a or the outer tube 21b.

The inner tube 21a may contain at least one selected from a group consisting of a polyamide resin, a polyester resin, a polyurethane resin, a polyolefin resin, a fluororesin, a vinyl chloride resin, a silicone resin, and natural rubber, or may contain at least one selected from a group consisting of a polyamide elastomer, a polyester elastomer, a polyurethane elastomer, a polyolefin elastomer, a vinyl chloride elastomer, and a silicone elastomer. The inner tube 21a may be made of only one of these materials, or two or more of these materials. The inner tube 21a preferably contains at least one selected from a group consisting of the polyamide resin, the polyolefin resin, and the fluororesin.

The inner tube 21a may optionally include a reinforcing member. Examples of the reinforcing member include a braided body made of a braided wire rod, and a coil body made of a spirally wound wire rod. This facilitates improvement in strength of the inner tube 21a.

Examples of the wire rod constituting the reinforcing member include metal wire and fiber. The metal wire is preferably made of a material such as stainless steel, titanium, a nickel-titanium alloy, a cobalt-chromium alloy, and a tungsten alloy. Among these, the stainless steel is more preferred. The metal wire may include a single wire or a twisted wire. Examples of the fiber include polyarylate fiber, aramid fiber, ultrahigh molecular weight polyethylene fiber, PBO fiber, and carbon fiber.

The fiber may be a monofilament or a multifilament.

The outer tube 21b may contain at least one selected from a group consisting of a polyamide resin, a polyester resin, a polyurethane resin, a polyolefin resin, a fluororesin, a vinyl chloride resin, a silicone resin, and natural rubber, or may contain at least one selected from a group consisting of a polyamide elastomer, a polyester elastomer, a polyurethane elastomer, a polyolefin elastomer, a vinyl chloride elastomer, and a silicone elastomer. The outer tube 21b may be made of only one of these materials, or two or more of these materials. The outer tube 21b preferably contains at least one selected from a group consisting of the polyamide resin, the polyolefin resin, and the polyurethane resin.

Examples of the linear body 21c include metal wire and resin wire. The metal wire has a distal end portion that is preferably coated with a resin (a so-called polymer jacket type) or a metal coil (a so-called coil jacket type), for easy improvement in flexibility at the distal end portion. The metal wire or the resin wire has an outer shape in a section in a thickness direction, examples of which include a circular shape, an elliptical shape, and a rectangular shape.

The metal wire or the metal coil is made of a material, examples of which include a shape memory alloy such as a nickel-titanium alloy, stainless steel, titanium, a cobalt-chromium alloy, and a tungsten alloy. The stainless steel is preferred among these.

Examples of the resin covering the resin wire or the metal wire include a polyamide resin, a polyester resin, a polyurethane resin, a polyolefin resin, a fluororesin, a vinyl chloride resin, a silicone resin, and natural rubber. The resin is preferably at least one selected from a group consisting of a polyamide elastomer, a polyester elastomer, a polyurethane elastomer, a polyolefin elastomer, a vinyl chloride elastomer, and a silicone elastomer.

As depicted in FIG. 1, the balloon catheter 20 has a proximal portion preferably provided with a handle 29. The handle 29 preferably has a lumen communicating with the inner tube 21a and extending in a longitudinal axis direction. The lumen can be utilized as an insertion path for guide wire or the like. Preferably, the handle 29 further includes a fluid injection portion 29a and has a lumen communicating with a flow path for fluid injection to the pressurization lumen 26 of the balloon 22.

The second lumen 2 has a proximal end portion preferably coupled with a negative pressure generator. The negative pressure generator exemplarily includes a pump. The negative pressure generator is configured to apply the negative pressure N to the second lumen 2. The negative pressure generator (not depicted) may be attached to a negative pressure generator attachment port 51 communicating directly or indirectly to a proximal end of the second lumen 2 of the sheath 5, as exemplarily depicted in FIG. 1.

As depicted in FIG. 4 and the like, preferably, the balloon 22 has the fixed portion 27 fixed to the shaft 21 and an unfixed portion 28 not fixed thereto, and the balloon catheter 20 includes a bar portion 25 extending distally from a distal end 28B of the unfixed portion 28.

The balloon catheter 20 including the bar portion 25 extending distally from the distal end 28B of the unfixed portion 28 facilitates detection of the peripheral bronchus 101. The bar portion 25 may include a distal end portion of the inner tube 21a, the distal fixed portion 27 of the balloon 22, and the bar body 25a as depicted in FIGS. 4 and 10 to 12, or may include the distal end portion of the linear body 21c, the distal fixed portion 27 of the balloon 22, and the bar body 25a as depicted in FIGS. 13 to 15. Alternatively, the bar portion 25 may not include the bar body 25a but may include the linear body 21c. In this case, the linear body 21c enables detection of the peripheral bronchus 101.

The bar body 25a made of a resin, metal wire, or the like having flexibility can easily avoid damage to the peripheral bronchus 101. Examples of the resin constituting the bar body 25a include a polyamide resin, a polyester resin, a polyurethane resin, a polyolefin resin, a vinyl chloride resin, a silicone resin, and natural rubber. The bar body 25a may be made of only one of these resins, or two or more of these resins. Among these resins, an elastomer resin is preferred because of its excellent flexibility. That is, the resin is preferably at least one selected from a group consisting of a polyamide elastomer, a polyester elastomer, a polyurethane elastomer, a polyolefin elastomer, a vinyl chloride elastomer, and a silicone elastomer.

The metal wire constituting the bar body 25a is made of a material, examples of which include a shape memory alloy such as a nickel-titanium alloy, stainless steel, titanium, a nickel-titanium alloy, a cobalt-chromium alloy, and a tungsten alloy.

The bar portion 25 has a length (mm) in a longitudinal axis direction, the length is preferably 0.5 times or more the outer diameter (mm) of the balloon 22 at the center 22C in the longitudinal axis direction of the shaft 21, and the balloon is pressurized with the pressurization lumen 26 being provided with the fluid. The balloon 22 being expanded and positioned closer to a terminal of the peripheral bronchus 101 is more likely to cause damage to the peripheral bronchus 101. However, the length being 0.5 times or more can easily avoid damage due to an excessive approach of the balloon 22 to the terminal of the peripheral bronchus 101. Accordingly, the length (mm) of the bar portion 25 in the longitudinal axis direction is more preferably 0.7 times or more the outer diameter (mm) of the balloon 22 at the center 22C in the longitudinal axis direction, further preferably 0.9 times or more, and still further preferably 1.0 time or more. Meanwhile, the length (mm) of the bar portion 25 in the longitudinal axis direction is preferably set to five times or less the outer diameter (mm) of the balloon 22 at the center 22C in the longitudinal axis direction, making it easier to expand a portion adjacent to the terminal of the peripheral bronchus 101. Accordingly, the length (mm) of the bar portion 25 in the longitudinal axis direction is preferably 5 times or less the outer diameter (mm) of the balloon 22 at the center 22C in the longitudinal axis direction, more preferably 3.5 times or less, and further preferably 3.0 times or less.

As depicted in FIG. 4, the bar portion 25 has a distal end portion 25b preferably including a curved portion having a curvature radius of 0.1 mm or more and 10 mm or less in a sectional view in the longitudinal axis direction. The curvature radius of 0.1 mm or more facilitates avoidance of damage to the peripheral bronchus 101. Accordingly, the curvature radius is more preferably 0.2 mm or more, and further preferably 0.4 mm or more. Meanwhile, the curvature radius of 10 mm or less facilitates insertion of the bar portion 25 to the peripheral bronchus 101. The curvature radius is thus preferably 10 mm or less, more preferably 8 mm or less, further preferably 6 mm or less, and still further preferably 2 mm or less.

The distal end portion 25b of the bar portion 25 has a shape not particularly limited, examples of which include a columnar shape, a hemispherical shape, a spherical shape, and a tapered shape. Among these, the hemispherical shape or the spherical shape is preferred due to easy avoidance of damage to the peripheral bronchus 101. The bar portion 25 has a portion other than the distal end portion 25b and having a shape, examples of which include a columnar shape, a polygonal columnar shape, and a columnar or polygonal columnar shape with a diameter gradually reduced toward a distal end. Among these, the columnar shape is preferred due to easy achievement of flexibility.

The bar body 25a may be constituted by a hollow member having a lumen penetrating in a longitudinal axis direction as depicted in FIGS. 10 and 13, or may be constituted by a solid member as depicted in FIGS. 11, 12, 14, and 15.

The lumen of the hollow member can be utilized as an insertion path for guide wire or the like, and the terminal of the peripheral bronchus 101 may be detected with use of the guide wire. Furthermore, a medicine such as a bronchodilator may be administered via the lumen. The hollow member is preferably joined to cover an outer side surface of the inner tube 21a or the linear body 21c of the shaft 21 as depicted in FIGS. 10 and 13.

The solid member facilitates transmission of a texture of the terminal of the peripheral bronchus 101 to the shaft 21. As depicted in FIGS. 11 and 14, the solid member has a proximal end portion that may be provided with a recess or a projection (not depicted) so as to be joined to the distal end of the inner tube 21a or the linear body 21c of the shaft 21. That is, the solid member may have the recess to be joined to cover the outer side surface of the inner tube 21a or the linear body 21c of the shaft 21, or may have the projection to be inserted and joined to the inner tube 21a of the shaft 21. Alternatively, the solid member may be joined to the fixed portion 27 of the balloon 22, without including the recess or the projection as depicted in FIGS. 12 and 15.

The balloon catheter 20 preferably has a bending load A₁ of 0.17 N or less upon pushing the bar portion 25 by 1.0 mm, and the bending load is obtained in accordance with a method of measuring a bending load as follows. The bending load A₁ of 0.17 N or less upon pushing the bar portion 25 by 1.0 mm facilitates avoidance of damage to the peripheral bronchus 101. The bending load A₁ upon pushing the bar portion 25 by 1.0 mm is more preferably 0.16 N or less, further preferably 0.15 N or less, and still further preferably 0.08 N or less. The bending load A₁ has a lower limit not particularly limited, but may be exemplarily 0.001 N or more.

### [Method of Measuring Bending Load]

There are prepared a lower block made of stainless steel and having a rectangular parallelepiped shape having a length in a longitudinal direction of 5 cm or more, a width perpendicular to the longitudinal direction of 2 cm or more, and a thickness of 2 cm or more, and an upper block made of stainless steel and having a rectangular parallelepiped shape having a length in a longitudinal direction of 5 cm or more, a width perpendicular to the longitudinal direction of 2 cm or more, and a thickness of 2 cm or more. The balloon catheter 20 is subsequently disposed between the lower block and the upper block such that the longitudinal axis direction of the shaft 21 is parallel to the longitudinal directions of the lower block and the upper block. Subsequently, a distal end of the lower block, a distal end of the upper block, and the distal end 28B of the unfixed portion 28 of the balloon 22 are aligned in the longitudinal axis direction of the shaft 21, and the balloon catheter 20 is fixedly sandwiched between the lower block and the upper block. Then measured is a load (N) upon pushing, by 1.0 mm in a direction perpendicular to the longitudinal axis direction of the shaft 21, a portion from the distal end of the bar portion 25 to a position distant by 1.0 mm in the longitudinal axis direction of the shaft 21 with use of a pressurizer having a rectangular pressurizing surface.

As depicted in FIG. 5, at the distal end 5B of the sheath 5, a distance between a center of the second lumen 2 and a center of the object lens 11 is preferably two times or less a diameter of the second lumen 2. This disposition facilitates removal of an impurity adhering to the object lens 11 upon sliding the distal end 5B of the sheath 5 sticking to the inner wall of the bronchus 100. The distance is more preferably 1.5 times or less, and further preferably 1.2 times or less. The distance has a lower limit that may be exemplarily 0.6 times or more.

As depicted in FIG. 5, at the distal end 5B of the sheath 5, the center of the second lumen 2 is preferably not positioned at a center of the sheath 5. This disposition allows a distal end 2B of the second lumen 2 to easily stick to a desired position by rotation or the like of the sheath 5 in the longitudinal axis direction.

At the distal end 5B of the sheath 5, the diameter (mm) of the second lumen 2 is preferably equal to or more than a diameter (mm) of the object lens 11. This configuration allows the distal end 5B of the sheath 5 to easily stick to the inner wall of the bronchus 100, as well as facilitates removal of an impurity adhering to the object lens 11. The diameter (mm) of the second lumen 2 is more preferably 1.2 times or more the diameter (mm) of the object lens 11, and further preferably 1.5 times or more. Meanwhile, the diameter (mm) of the second lumen 2 may be 3.0 times or less the diameter (mm) of the object lens 11, or may be 2.5 times or less.

At the distal end 5B of the sheath 5, assuming that an area of a region surrounded with an outer circumference of the sheath 5 has 100 area percent, the second lumen 2 has an area ratio that is preferably 10 area percent or more. This configuration improves sticking force and allows the distal end 5B of the sheath 5 to easily stick to the inner wall of the bronchus 100. The area ratio is more preferably 15 area percent or more, and further preferably 20 area percent or more. Meanwhile, the area ratio has an upper limit not particularly limited, and may be 80 area percent or less, or may be 60 area percent or less.

In the longitudinal axis direction of the sheath 5, the object lens 11 has a distal end preferably positioned identically with the distal end 5B of the sheath 5 as depicted in FIG. 2 or positioned distally to the distal end 5B of the sheath 5. This disposition facilitates wiping an impurity in the bronchus 100 adhering to the object lens 11. A distance between the distal end of the object lens 11 and the distal end 5B of the sheath 5 in the longitudinal axis direction of the sheath 5 is preferably 5 mm or less, more preferably 3 mm or less, and further preferably 1 mm or less. This disposition allows the distal end 5B of the sheath 5 to easily stick to the inner wall of the bronchus 100.

As depicted in FIG. 5, the sheath 5 may have a third lumen 3 having the longitudinal axis direction X, and may further have a fourth lumen 4 having the longitudinal axis direction X. The third lumen 3 may be provided therein with a first illumination lens 30, and the fourth lumen 4 may be provided therein with a second illumination lens 40. This facilitates observation of the peripheral bronchus 101. The first illumination lens 30 and the second illumination lens 40 are preferably fixed to the third lumen 3 and the fourth lumen 4, respectively, so as not to move in the longitudinal axis direction X.

The lumens other than the second lumen 2 are preferably sealed at the distal end 5B of the sheath 5. This facilitates improvement in sticking force in the second lumen 2.

The sheath 5 has an outer side surface preferably provided with no through hole communicating with the second lumen 2 from the distal end 5B of the sheath 5 to a position distant by 1 cm in the longitudinal axis direction. This facilitates improvement in sticking force of the second lumen 2. Furthermore, the outer side surface of the sheath 5 from the distal end 5B of the sheath 5 to a position distant by 3 cm in the longitudinal axis direction is more preferably provided with no through hole communicating with the second lumen 2, and the outer side surface of the sheath 5 from the distal end 5B to a proximal end of the sheath 5 is further preferably provided with no through hole.

As depicted in FIG. 1, the sheath 5 has a proximal portion preferably provided with an operation portion 55 incorporating the proximal portion of the sheath 5. The operation portion 55 thus provided allows an operator to grip the operation portion 55 for adjustment of an insertion angle or the like of the sheath 5. Examples of the operation portion 55 include a resin case.

The proximal portion of the sheath 5 may be optionally divided into two or more branches. In an exemplary case where the proximal portion of the sheath 5 is divided into two branches, the proximal end of the second lumen 2 at a first one of the branches may be coupled to the negative pressure generator attachment port 51 at the operation portion 55, and the proximal end of the second lumen 2 at a second one of the branches may be coupled to the insertion hole 52 at the operation portion 55. Alternatively, the proximal portion of the sheath 5 may not be branched. In this case, the proximal end of the second lumen 2 may be coupled to the negative pressure generator attachment port 51, and there may be provided a through hole from the second lumen 2 to the outer side surface so as to communicate with a passage reaching the insertion hole 52 in the operation portion 55. Alternatively, the proximal end of the second lumen 2 may be coupled to the insertion hole 52, and there may be provided a through hole from the second lumen 2 to the outer side surface so as to communicate with a passage reaching the negative pressure generator attachment port 51 in the operation portion 55.

The insertion hole 52 is preferably provided with a sealing member configured to seal the insertion hole 52 when the negative pressure N is applied to the second lumen 2. This facilitates improvement in sticking force of the second lumen 2. Examples of the sealing member include a forceps plug provided with a cut, and specifically include a silicon rod having a cut for a Y connector.

The present invention further includes a method of using the bronchoscope 50. Specifically, the method of using the bronchoscope 50 includes applying the negative pressure N to the second lumen 2 to cause the distal end 5B of the sheath 5 to stick to the inner wall of the bronchus 100.

The method of using the bronchoscope 50 preferably includes sliding the distal end 5B of the sheath 5 sticking to the inner wall of the bronchus 100, to remove an impurity in the bronchus 100 adhering to a distal end of the endoscopic camera 10. This facilitates observation of the peripheral bronchus 101. Examples of the impurity include a secretion from the bronchus 100, blood, and foreign matter coming from outside the bronchus.

Sticking to the inner wall of the bronchus 100 is preferably executed while negative pressure is applied to the pressurization lumen 26 of the balloon 22. This easily prevents expansion of the balloon 22 due to application of the negative pressure N to the second lumen 2.

When sticking to the inner wall of the bronchus 100, the negative pressure N is applied to the second lumen 2 preferably provided therein with the balloon 22. This allows the balloon 22 to be distally extruded from the distal end 2B of the second lumen 2 immediately after removal of dirt on the endoscopic camera 10 through sticking and sliding.

The method of using the bronchoscope 50 preferably includes: distally extruding the balloon 22 from the distal end 2B of the second lumen 2; applying positive pressure P to the balloon 22 to expand the balloon 22; applying negative pressure to the balloon 22 to contract the balloon 22; pulling back the balloon 22 into the second lumen 2; and observing with use of the endoscopic camera 10. When the bronchoscope 50 is inserted to the peripheral bronchus 101 in a state where the peripheral bronchus 101 is expanded by the balloon 22 and the balloon 22 is contracted, insertion resistance is reduced for easy insertion.

When applying the positive pressure P to the balloon 22 to expand the balloon 22, a distance (mm) from the distal end 5B of the sheath 5 to a proximal end 28A of the unfixed portion 28 of the balloon 22 upon application of the positive pressure P is preferably three times or less a distance (mm) from the proximal end 28A to the distal end 28B of the unfixed portion 28 of the balloon 22. This facilitates insertion before contraction of the peripheral bronchus 101 thus expanded.

The method of using the bronchoscope 50 preferably includes distally pushing the bronchoscope 50 after the balloon 22 is pulled back into the second lumen 2. When the balloon 22 is pulled back into the second lumen 2, the bronchoscope 50 can be easily pushed into the peripheral bronchus 101.

Each of these steps is not necessarily executed only once, and may be repetitively executed twice or more. For example, after distally extruding the balloon 22 from the distal end 2B of the second lumen 2, applying the positive pressure P to the balloon 22 to expand the balloon 22, applying negative pressure to the balloon 22 to contract the balloon 22, pulling back the balloon 22 into the second lumen 2, and distally pushing the bronchoscope 50, the same steps may be executed again.

The present application claims benefit of priority based on Japanese Patent application No. 2019-189683 filed on Oct. 16, 2019. The entire contents of the specification of Japanese Patent application No. 2019-189683 filed on Oct. 16, 2019 are incorporated in the present application for reference.

### DESCRIPTION OF REFERENCE SIGNS

1 first lumen
2 second lumen
2B distal end of second lumen
3 third lumen
4 fourth lumen
5 sheath
5B distal end of sheath
10 endoscopic camera
11 object lens
12 image transmitter
20 balloon catheter
21 shaft
21a inner tube
21b outer tube
21c linear body
21B distal end of inner tube
21C distal end of linear body
22 balloon
22C center of balloon in longitudinal axis direction of shaft
23 straight tube portion
23A proximal end of straight tube portion
23B distal end of straight tube portion
24 tapered portion
25 bar portion
25a bar body
25b distal end portion of bar portion
25B distal end of bar body
26 pressurization lumen
27 fixed portion
27B distal end of fixed portion
28 unfixed portion
28A proximal end of unfixed portion
28B distal end of unfixed portion
29 handle
29a injection portion
30 first illumination lens
40 second illumination lens
50 bronchoscope
51 negative pressure generator attachment port
52 insertion hole
53 connector portion
55 operation portion
100 bronchus
101 peripheral bronchus

1. A bronchoscope comprising:
a sheath having a longitudinal axis direction, the sheath having a first lumen and a second lumen each extending in the longitudinal axis direction;
an endoscopic camera disposed in the first lumen; and
a balloon catheter including a shaft and an inflatable balloon provided at a distal portion of the shaft, the balloon catheter being disposed in the second lumen so that the balloon catheter is movable in the longitudinal axis direction, wherein the inflatable balloon is configured to be inflated by applying positive pressure therein, and
the second lumen is configured so that a distal end of the sheath is capable of sticking to an inner wall of a bronchus by applying a negative pressure to the second lumen.

2. The bronchoscope according to claim 1, wherein the inflatable balloon is configured so that when the balloon is inflated, the inflated balloon has an outer diameter (mm) at a center in a longitudinal axis direction of the shaft, the outer diameter being larger than an outer diameter (mm) of the sheath at the distal end of the sheath.

3. The bronchoscope according to claim 1, wherein the inflatable balloon is configured so that when the balloon is inflated, the inflated balloon includes a straight tube portion, and, the straight tube portion has a length from a proximal end to a distal end in a longitudinal axis direction of the shaft, the length being equal to or more than the outer diameter of the balloon at the center in the longitudinal axis direction of the shaft.

4. The bronchoscope according to claim 1, wherein the inflatable balloon is configured so that when the balloon is inflated, the inflated balloon includes a spherical portion or a prolate spherical portion, and the spherical portion or the prolate spherical portion has a length from a proximal end to a distal end in a longitudinal axis direction of the shaft, the length being equal to or more than the outer diameter of the balloon at the center in the longitudinal axis direction of the shaft.

5. The bronchoscope according to claim 1, wherein in a section perpendicular to the longitudinal axis direction of the sheath, the second lumen has an area being 1.2 times or more an area of a region surrounded with an outer circumferential line of the balloon being contracted.

6. The bronchoscope according to claim 1, wherein the second lumen has a proximal end portion connected to a negative pressure generator.

7. The bronchoscope according to claim 1, wherein the inflatable balloon has a fixed portion fixed to the shaft and an unfixed portion not fixed thereto, and the balloon catheter includes a bar portion extending distally from a distal end of the unfixed portion.

8. The bronchoscope according to claim 7, wherein the bar portion has a length in a longitudinal axis direction, the length being 0.5 times or more the outer diameter of the balloon at the center in the longitudinal axis direction of the shaft, the balloon being inflated.

9. The bronchoscope according to claim 7, wherein the bar portion has a distal end portion including a curved portion having a curvature radius of 0.1 mm or more and 10 mm or less in a sectional view in a longitudinal axis direction.

10. The bronchoscope according to claim 1, wherein the balloon is a noncompliant balloon or a semicompliant balloon.

11. A method of using the bronchoscope according to claim 1, the method comprising applying negative pressure to the second lumen to cause the distal end of the sheath to stick to the inner wall of the bronchus.

12. The method of using the bronchoscope according to claim 11, the method comprising sliding the distal end of the sheath sticking to the inner wall of the bronchus, to remove an impurity in the bronchus adhering to a distal end of the endoscopic camera.

13. The method of using the bronchoscope according to claim 11, wherein sticking to the inner wall of the bronchus is executed while negative pressure is applied to the balloon.

14. The method of using the bronchoscope according to claim 11, the method comprising:
distally extruding the inflatable balloon from a distal end of the second lumen;
applying positive pressure to the balloon to expand the balloon;
applying negative pressure to the balloon to contract the balloon;
pulling back the balloon into the second lumen; and
observing with use of the endoscopic camera.

15. The method of using the bronchoscope according to claim 14, the method comprising distally pushing the bronchoscope after the balloon is pulled back into the second lumen.
